(19) United States
(12) Patent Application Publication
(10) Pub. No.: US 2002/0025337 A1
(43) Pub. Date: Feb. 28, 2002

(54) LIPID VEHICLE DRUG DELIVERY COMPOSITION CONTAINING VITAMIN E

(76) Inventors: LISBETH ILLUM, NOTTINGHAM (GB); CLIVE WASHINGTON, NOTTINGHAM (GB); SIMON LAWRENCE, WEST SUSSEX (GB); PETER WATTS, NOTTINGHAM (GB)

Correspondence Address: Patrea L Pabst, HOLLAND & KNIGHT LLP, One Atlantic Center, 1201 West Peachtree Street Ste.2000, Atlanta, GA, 30309-3400, US

(21) Appl. No.: 09/000,039

(22) PCT Filed: Jul. 22, 1996
(86) PCT No.: PCT/GB96/01758

(30) Foreign Application Priority Data
Jul. 20, 1995 (GB) 9514878.9

(51) Int. Cl. A61K 9/127

(52) U.S. Cl. 424/450

(57) ABSTRACT

The present invention provides a drug delivery composition comprising a lipid vehicle containing a drug and Vitamin E to enhance the solubility of the active drug in the lipid vehicle. The composition is particularly useful for drugs which are poorly soluble. The composition may be in the form of a liposome or an oil-in-water emulsion. The Vitamin E may be mixed with a pharmaceutically acceptable oil such as a marine oil or a vegetable oil.

The present invention relates to a drug delivery composition comprising a lipid vehicle, and more particularly to a drug delivery composition comprising vitamin E to enhance the solubility of the drug in the lipid vehicle.

Drugs can be administered by a variety of routes to include oral and parenteral. It is often useful to have available an injectable form of a drug in order to provide rapid onset of action, or direct delivery to its site of action via the blood stream. A number of drugs are difficult to administer by injectable routes because they are poorly soluble in water such that an excessive volume of solution would be required in order to deliver a dose of the compound. Various approaches known to those skilled in the art can be used in order to increase the loading of the drug in the parenteral product. These include the use of cosolvents, surfactants, liposomes and emulsions.

Emulsion systems have long been used for pharmaceutical purposes. Such systems include oil-in-water emulsions, water-in-oil emulsions and more complex systems known as multiple emulsions. Microemulsions that comprise thermodynamically stable systems which are normally transparent are also well known to those skilled in the art. Oil-in-water emulsions, where the continuous phase is aqueous and the disperse phase is oily in nature, can be used for a variety of purposes and administered via a variety of routes to include injection as well as administration to the eye, nose, lung, gastrointestinal tract or vagina.

Parenteral emulsions have an important role in drug delivery, diagnosis and in nutrition. The subject has been well reviewed in the literature, see for example Davis et al In “Encyclopedia of Emulsion Technology”, Becher P. Ed., Dekker, New York, Vol. 2 pp 159-238, Benita S. and Levy M. Y., J. Pharm. Sci. 82 1069 (1993) Davis S. S. et al, Ann N.Y. Acad. Sci. 507 75 (1987), and Singh M. and Ravin, L.J., J. Parent. Sci. Tech. 40-34 (1986). Emulsions given intravenously for the purposes of drug delivery have been well described in various patents for example, U.S. Pat. Nos. 4,168,308, 4,647,586, 4,816,247, EP 0331755 and EP 0321429. The oil phase is used to carry the drug substance of interest, either with the drug dissolved in the oil or with the drug carried in the interfacial layer surrounding the oil droplets or a combination of both effects. The oil phases of choice are usually based upon pharmaceutically acceptable vegetable oils such as soya bean oil, olive oil, sesame oil, safflower oil, or more recently fractionated oils known as “medium chain triglycerides”. The chosen emulsifier is one that is non-toxic and acceptable to regulatory authorities. Egg phospholipid (egg lecithin) has been a material of choice. The block copolymers known as the poloxamers (Pluronic trade name) have also been employed in experimental formulations. Products that have reached the market place include emulsions containing the anaesthetic agent di-isopropylphenol (propofol) and the sedative diazepam. Other emulsifying agents include bile salt derivatives as described in EP 0391369.

An emulsion delivery system for lipid soluble drugs has many advantages over other approaches such as those based upon high concentrations of surface active agent where the drug is essentially solubilised into a micellar phase or system based upon cyclodextrins. The latter, especially hydroxypropylbeta cyclodextrin, can provide an enhanced solubilisation of drugs. However, such cyclodextrins are known to be associated with toxicity problems particularly with regard to their effect on the kidney. Emulsion systems often have advantages over liposomes (phospholipid vesicles) in that they have a higher carrier capacity for lipid soluble materials.

One problem with emulsion systems can be the poor loading of the drug into the oil phase or onto the surface of oil droplets. Such a loading is dependent upon the properties of the oil and the properties of the drug. It has been found that highly lipid soluble drugs (those with an octanol/water partition coefficient greater than 1 million) are normally well dissolved in the oil phase and satisfactory emulsions can often be prepared. However, many drugs of pharmaceutical interest do not have such a high lipid solubility or have very high melting points that result in poor solubility in the small number of pharmaceutically acceptable non-aqueous solvents. Drugs with poor lipid solubility can be given an enhanced lipid nature through the formation of prodrugs. Usually an ester linkage or a carbamate linkage is preferred because such linkages can be easily cleaved in the body to release the parent drug from the prodrug moiety. This approach has been adopted with success with the anticancer drug mitomycin. Lutz et al. J. Pharm. Pharmacol. 45, Suppl. p 59, 1992. However, such approaches result in the chemical modification of the drug and necessitate additional and expensive toxicological evaluation.

In many situations, it is desirable to have drug loadings in emulsion systems of at least 1 mg/ml or even higher. (All solubilities quoted herein are at room temperature (25° C.) unless otherwise stated.) This allows a sufficient quantity of drug to be given in a minimum quantity of emulsion system. The dispersed phase of the emulsion can be increased from 5-10% to 20-30% and even to 40-50% volume in order to provide high loading where the drug is dissolved in the oil phase. Unfortunately, the greater quantity of oil employed causes the emulsion to be viscous and also leads to the administration of substantial quantities of the oil phase (eg. triglycerides) which may have metabolic consequences. Such emulsions are also hard to homogenize and the resultant particle size can be large. Thus, it is desirable in many situations to have a high drug loading in an emulsion system with a minimum volume of injected material (both oil and water phases).

It is known in the art that the solubility of many drugs in vegetable oils can be low, even though such compounds can display high octanol-water partition coefficient. In such cases the solubility of the drug in oil can sometimes be increased by the use of oil mixtures. For example it has been shown in previous examples in the patent literature that acetylated monoglycerides as well as acetylated diglycerides can be used to improve the solubility of drugs in the oil phase of an oil-in-water emulsion, (U.S. Pat. No. 4,168,308, Hogskilde et al, Anaesthesia 42 1045, (1987)). Moreover, the marketed emulsion products of diazepam contain both soya bean oil and acetylated monoglyceride as the disperse phase.

Liposomal Itraconazole systems have been reported in WO 93/15719. The auxiliary formulating agents demethylisosorbide and tetraglycol are used.

The difficulties of producing parenteral formulations of taxol are well described in the prior art. For example, Tarr et al. Pharm. Res. 4, 162 (1987) described an oil in water emulsion system containing 50% triacetin as an alternative system to alcohol: surfactant mixtures where the solubility of the drug in the vehicle did not exceed 0.6 mg/ml. Unfortunately their emulsion. while having a good loading of the drug, had poor pharmaceutical characteristics. The presently available commercial taxol formulations often contain large quantities of surfactant such as ethoxylated castor oil materials. These are known to be associated with undesirable side effects such as anaphylaxis. The product needs to be diluted before use.

WO 93/136391 (EP 539215A1) and JP06092856-A refer to the use of Vitamin E as an absorption enhancing agent for the better percutaneous absorption of drugs. Vitamin E is stated to enhance the penetration of therapeutically active agents and can also act as a carrier. No mention is made of the possible use of Vitamin E (or derivatives thereof) in emulsions. Liposomal Vitamin E systems are also known (Surtres et al J. Pharm. Pharmac. 45 514, (1993), Halks-Miller et al., Lipids 20 195 (1985), Urano et al. Archiv. Biochem. Biophys. 303 10 (1993).

Kato et al have described the blood clearance and tissue distribution of various formulations of alpha tocopherol injection after intravenous administration. Liposomal and emulsion systems are mentioned but no consideration is given to drug delivery of compounds such as antifungal agents and anticancer agents (Kato, Y. et al. Chem. Pharm. Bull. 41 599, 1993).

Vitamin E emulsions for use in drug therapy, for example as a vitamin preparation and as a therapeutic agent in cancer treatment, have been described previously (WO 94/21232. EP 599543). In such emulsions, the Vitamin E has been the active material and has not been used as an excipient to solubilise a poorly soluble drug.

U.S. Pat. No. 5,364,631 describes the preparation of tocopherol ester liposome preparations at acid pH containing a bioactive agent. The composition is used for bioactive agents requiring or tolerating low pH conditions. Tocopherol hemisuccinate is a favoured material for liposome formation.

We have now surprisingly found that drugs which exhibit a low solubility in oils, especially vegetable oil, can demonstrate high solubilities in Vitamin E, and that Vitamin E can be used to enhance the drug solubility of poorly oil soluble drugs in a lipid vehicle drug delivery composition.

The present invention therefore provides a drug delivery composition comprising a lipid vehicle containing a drug and Vitamin E to enhance the solubility of the active drug in the lipid vehicle.

The term “Vitamin E” as used herein is used to include all tocol and tocotrienol derivatives that exhibit Vitamin E activity.

The nomenclature for Vitamin E and related compounds is unclear in current practice and can vary when used by different compendia and organisations. This problem has been well addressed by Sheppard et al. in Vitamin E in Health and Disease, Editors Packer, L. and Fuchs, J. Dekker, New York 1993 p.9.

The United States Pharmacopoeia described Vitamin E as a form of α-tocopherol. This includes d- or d, 1, α tocopherol, d- or d, 1-α-tocopherol acetate and d- or d, 1-α-tocopherol succinate. The Association of Official Analytical Chemists (AOAC) states that the term Vitamin E should be used as a generic description for all tocol and tocotrienol derivatives that exhibit Vitamin E activity. Thus the term tocopherols is synonymous with Vitamin E but also for methyl tocols. α-tocopherols is a trivial name without defined steriochemistry.

Tocopherol is an approved material for parenteral administration and is present in lipid emulsion systems comprising multivitamins. Vitamin E is also present in fat emulsion products for drug delivery but solely as an antioxidant at low concentrations, and certainly less than 1% of the total emulsion. A typical example can be found in the paper by Mbela et al. Int. J. Pharm. 110 189 (1994) where tocopherol is added to an emulsion formulation as an antioxidant at a concentration of 0.02%. Tocopherol is a particularly interesting material in that it can be given in high doses orally, up to 3500 mg on a daily basis for many days. The tolerance and. safety of Vitamin E has been described by Kappus and Diplock, Free Radical Biology and Medicine 13 55 (1992), Tomassi and Silana, Fd. Chem. Toxic. 24 1051 (1886). Bendich and Machlin have reviewed the clinical studies from 1986 to 1991 on the safety of Vitamin E in the monograph entitled Vitamin E in Health and Disease, Edited by Packer and Fuchs, Dekker, New York, 1993, p 411. For orally administered Vitamin E it is expected that 50-70% of this dose will be taken into the systemic circulation following transport through the lymphatic system. The oral uptake of Vitamin E and the effect of formulation factors is reviewed by Charman in Lymphatic Transport of Drugs, Editors, Charman and Stella, Chapter 4, CRC Press, 1992.

The Vitamin E is preferably in the form of the free alcohol, but suitable tocopherol derivatives are esters of tocopherol such as the linoleate, nicotinate, acetate or acid succinate ester.

We use the term drug to include all compounds which it may be desired to administer to a mammal and which are pharmaceutically, pharmacologically, therapeutically, diagnostically, cosmetically or prophylactically active or which are a prodrug for such a compound. Preferably, the drug is not a vitamin or dietary mineral such as zinc or iron.

The drug should have reasonable solubility in Vitamin E. Preferably, the drug has a solubility of at least 1 mg/ml and more preferably at least 5 mg/ml in Vitamin E.

The ability of a drug to dissolve in sufficient quantities in Vitamin E for use in the invention will depend on the affinity of the drug for this oil. We have discovered that those drugs that are poorly soluble in chlorinated organic solvents such as chloroform are also poorly soluble in Vitamin E. In contrast drugs that have good solubility in chloroform have acceptable solubility in Vitamin E. Suitable drugs preferably have a solubility in chloroform of 6 mg/ml or more, preferably, 10 mg/ml or more.

In contrast if the molecule demonstrates good solubility in methanol (more than 10 mg/ml) it will demonstrate low solubility in Vitamin E (less than 1 mg/ml). Thus a person skilled in the art will be able to decide whether a drug is a suitable candidate for the invention by reviewing published data on solubility in chloroform or methanol. We have defined a new parameter, the SVE (“solubility in Vitamin E”) ratio, to help in this regard. This is defined as the solubility (mglml) in chloroform divided by the solubility (mg/ml) in methanol.

Those drugs with SVE greater than 10 are preferred and those materials with SVE greater than 100 are especially preferred. Some representative values are given in Table 1.

To measure the SVE of a drug, saturated solutions of the drug are prepared in methanol and chloroform. An appropriate means of preparing a saturated solution is to suspend approximately 60 mg of drug in 3 ml of the solvent and stir for 24 hours at room temperature. If all of the drug dissolves during this time, further 10 mg aliquots should be added until a suspension is again formed. After 24 hours, the suspension are centrifuged or filtered to separate drug in solution from undissolved particulate drug. The drug solutions are assayed for drug content by an appropriate means, eg. high performance liquid chromatography, and the saturated solubility calculated. The SVE is calculated by dividing the solubility in chloroform by the solubility in methanol and expressed as weight in volume (w/v).

By way of example, the SVE of itraconazole was measured as follows.

Into each of two 10 ml bottles was weighed 60 mg of itraconazole. To one bottle was added 3 ml of chloroform. The itraconazole instantly dissolved. Further itraconazole was added in 100 mg aliquots until a suspension was formed. A total of 1360 mg of itraconazole was added. To the other bottle was added 3 ml of methanol. A magnetic stirrer bar was added to each bottle and the contents were left to stir on a magnetic stirrer. After being left overnight to stir (18 hours), the contents of each bottle were filtered through a 0.1 μm PTFE filter (Whatman). To assay the chloroform filtrate for itraconazole content, 0.2 ml was diluted to 1000 ml with a mixture of 95% of 0.01M tetrabutyl ammonium hydrogen sulphate solution in water/5% acetonitrile. To assay the methanol filtrate for itraconazole content, 1 ml was diluted to 5 ml with the same diluent used for the chloroform analysis. Both samples were assayed for itraconazole content using reverse-phase high performance liquid chromatography. The concentrations of itraconazole in the diluted chloroform and methanol filtrates were 70 μg/ml and 140 μ/ml respectively. Thus, the solubilities of itraconazole in chloroform and methanol were 350 mg/ml and 0.7 mg/ml respectively. Therefore, the SVE value for itraconazole was 350/0.7=500.

The suitability of a drug may also be determined by measuring its solubility parameter.

The ability of solvents to mix well together or for solvents to dissolve in solvents can be estimated using the procedure of solubility parameters. This method, based upon concepts of cohesion density, was developed originally by Hildebrand and refined by others for a wide range of materials. The concepts of solubility parameters is well reviewed by Barton in CRC Handbook of Solubility Parameters and Other Cohesion Parameters, 2nd Ed. CRC Press, 1991. Those skilled in the art often use this concept to estimate whether a particular drug (solute) will dissolve in a given solvent and the extent of such solubility. In order to do this the solubility parameter of the solute and solvent are required. While solubility parameter values are available for many solvents used in pharmaceutical formulations, solubility parameter values for drugs are not normally available. However, methods have been established wherein solubility parameter values can be calculated. The procedure described by Fedors is well known in this regard (Polym. Eng. Sci. 14 147, 1974).

It has been established previously that polar drug materials have solubility parameters greater than 13 and non-polar materials below 8. The solubility parameter for Vitamin E is estimated to be 9.7, similar to the value for chloroform (9.2). Methanol has a solubility parameter of 14.7. These drugs that have solubility parameter values close to that of Vitamin E would be expected to show acceptable solubility in Vitamin E. Values for the drugs listed in Table 1 are calculated according to the method of Fedors (vide infra).

TABLE 1
Solubility of drugs in organic solvents and Vitamin E

| | | | Solubility (mg/ml) | | SVE* | Solubility** |
|---|---|---|---|---|---|---|
| Drug | Water | Methanol | Chloroform | Vitamin E | Parameter | Parameter |
| Itraconazole | insol | insol | 500 | 60 | >1000 | 10.6 |
| Taxol | insol | 0.03 | 6 | 11 | 200 | 11.9 |
| Cyclosporin | Sl. sol. | 0.71 | 363 | 100 | 520 | 10.7 |
| Ergosterol | insol. | 1.5 | 32 | 50 | 25 | 9.6 |
| Cholesterol | insol. | 5 | 200 | 150 | 40 | 9.6 |
| Prednisolone | 0.22 | 33 | 5.0 | insol. | 0.02 | 13.6 |
| Amphotericin | insol. | sol. | insol. | insol. | <1 | 14.4 |

*SVE- ratio of solubility in chloroform to that in methanol.
**as calculated by method of fedors (1974) Polymer Engineer. Sci. 14, 147, incorporated herein by reference.
Sl. sol - slightly soluble.

For the present invention we prefer drugs that have a solubility parameter value between 8 and 13 and more especially between 9 and 12. The same concepts can be applied if derivatives of Vitamin E are used. For example corresponding solubility parameters are

| | |
|---|---|
| Vitamin E acetate | 9.1 |
| Vitamin E quinone | 8.6 |

Drugs that are especially suitable for the emulsion formulation are antifungal agents such as itraconazole, anticancer agents such as taxol, hexamethylmelamine, penclomedine and lipophilic porphyrin derivatives, steroids such as pregnanolone, anaesthetic agents such as propofol (diisopropyl phenol), retinoid compounds, cardiovascular agents such as S-emapomil, agents such as prostaglandins, lipophilic peptides such as cyclosporin, and protein kinase C inhibitors such as dihydrosphingasine.

It is preferred if the drug loading is at least 0.1 mg/ml, more preferably 1 mg/ml and still more preferably 10 mg/ml.

The composition may be in the form of a liposome or more preferably an emulsion, advantageously an oil-in-water emulsion. The Vitamin E should be present in the liposome emulsion in a concentration of at least 1% in the disperse phase, preferably at least 5% and more preferably at least 10%.

The Vitamin E may be provided as a mixture with a pharmaceutically acceptable oil. This includes oils that can be used in an emulsion formulation which will be administered parenterally, orally, nasally, vaginally and rectally, as well as into the eye or lungs. Such oils include vegetable oils such as soybean oil, sesame oil, safflower oil, castor oil, corn oil and olive oil, as well as marine oils such as cod liver oil and sardine oil. Oils such as squalene and squalane could also be used. In all cases the choice of the oil will be dictated by the route of administration and the metabolic character of the oil. Such mixtures of Vitamin E and vegetable oil can be readily emulsified with phospholipid emulsifiers. Vitamin E above is difficult to emulsify with a phospholipid emulsifier on its own. Suitable phospholipid emulsifiers are egg phospholipids. Any other known emulsifying agent may also be used, such as a non-ionic surfactant.

In the simplest form, an oil-in-water emulsion contains three components: an oil phase, an aqueous phase and a stabiliser. The emulsion is prepared by dissolving the stabiliser in the aqueous phase. The aqueous phase is then mixed with the oil phase to form a dispersion of oil droplets. The size and size distribution of the oil droplets will depend on the method of mixing. In stable emulsions, the droplet size generally lies in the range 0.1-10 μm. High shear mixing using equipment such as an homogeniser or a microfluidiser is the preferred method of preparing pharmaceutical emulsions. For good emulsion stability, the oil phase should comprise between 10 and 60% of the total emulsion volume. In theory, the oil phase can comprise a maximum of 74% of the total emulsion volume of an oil-in-water emulsion.

The emulsions may be administered orally, or parenterally. We use the term parenterally to include administration to the muscles, subcutaneous tissue, peritoneal cavity, venous system, arterial system, lymphatic system, spinal fluid (intrathecal, epidural) and joint cavities. Parenteral formulations will be sterile and usually pyrogen-free.

The emulsion can also be administered to the gastrointestinal tract or other mucosal surfaces such as the eye, nose, vagina or rectal cavity. When formulating an emulsion for a particular route, the person skilled in the art will appreciate that the choice of emulsifier that will be employed to form a pharmaceutically stable system will be dictated by considerations of toxicity and regulatory acceptance. Thus for a parenteral emulsion and an emulsion administered to delicate surfaces such as eye, vagina or nose, the emulsifier could be a phospholipid or non-ionic surfactant in the form of a block co-polymer (Poloxamer 188). For administration to the gastrointestinal tract a wider choice of emulsifier is available to include non-ionic surfactants of different types as well as ionic emulsifiers and natural gums.

Examples include sodium oleate, triethanolamine oleate, polyoxyethylene sorbitan monooleate, polyoxyethylene sorbitan monolaurate, gum acacia, gelatin, methylcellulose and gum tragacanth.

When the emulsion intended for injection contains a high content of Vitamin E in the oil phase (that is greater than 50%) the preferred emulsifier is a block copolymer such as poloxamer 188.

The emulsion of the invention is useful for oral administration. Itraconazole and similar drugs are known to have a poor and variable bioavailability from the gastrointestinal tract largely, because of the poor solubility of the drug in the fluids of the stomach and intestine. By preparing a self-emulsifying or well solubilised oily formulation of the drug where Vitamin E is used as a drug solubilizing agent, the oral absorption of itraconazole has been improved. This approach greatly minimises the volume of oil vehicle required as compared to a conventional emulsion prepared from vegetable oils or even fish (marine) oils. Thus, by dissolving itraconazole in Vitamin E, it has been found possible to produce a more reliable and convenient oral formulation. A spontaneously emulsifying system can be prepared using various methodologies prescribed in the prior art.

By mixing the solution of drug in Vitamin E with a pharmaceutically acceptable oil-in-water emulsifier, a formulation can be prepared which readily disperse in contact with aqueous media. Such emulsifiers should be miscible with Vitamin E and include, but are not limited to, polyoxyethylene sorbitan fatty acid esters (eg. Tween®), polyoxyethylene alkyl ethers (eg. Brij®) and polyglycolized glycerides (eg. Labrasol®, Gattefosse). Such formulations are suitable for filling into pharmaceutical capsules made of materials such as gelatin or starch.

Surfactant systems that have been approved by regulatory authorities are well known. In the gastrointestinal tract the system can emulsify spontaneously because of the low interfacial tension created by the addition of the emulsifier and co-surfactant systems.

Thus, by using Vitamin E, either alone or mixed with a pharmaceutically acceptable oil, we have found that the solubility of certain poorly soluble drugs in a lipid system can be greatly increased.

In particular in studies on the preparation of emulsions of the antifungal drug itraconazole and the anticancer agent taxol, we have found that the substances have a surprisingly high solubility in Vitamin E. These drugs themselves are poorly soluble in conventional vegetable oils such as soya bean oil. For example for itraconazole and taxol the solubility of the drug in vegetable oils is 400 microgram/ml or less. Similar values are found with other vegetable oils such as sesame oil and fractionated medium chain triglycerides. However, the drugs display solubility in Vitamin E (tocopherol). This represents a most surprising increase in solubility compared to a simple triglyceride vegetable oil. We have also found that it is possible to mix vegetable oil with Vitamin E and thereby provide various levels of enhanced solubility of drugs so that they may be administered parenterally as an emulsion formulation.

Emulsions containing various quantities of soy bean oil mixed with tocopherol and added intraconazole have been prepared. In this way we have been able to prepare emulsions containing 2 mg/ml of the antifungal agent Itraconazole This is much higher than has been hitherto reported for non Vitamin E formulations. We have also been able to prepare liposomal systems with a high drug loading by dissolving Itraconazole in Vitamin E and then making a liposomal product using conventional procedures. Similarly emulsion products containing a loading of the anticancer drug taxol at a quantity of at least 1 mg/ml have been prepared. Once again this is a higher concentration of drug in a vegetable oil based emulsion that has been reported hitherto.

The liposome formulation can be prepared according to well established methods known to those skilled in the art (for example see Chapter 1, Preparation of liposomes, in Liposome drug delivery systems, Betageri et al., Technomic Publishing Co. 1993).

Liposomes of different structures, namely multilamellar vesicles, small unilamellar vesicles and large unilamellar vesicles, can be produced. The basic constituent is a phospholipid derived from both natural and synthetic sources. The main material is phosphatidylcholine but other neutral and charged lipids can be included. Cholesterol can also be added.

The traditional way to produce liposomes is to dissolve the constituent lipids in an organic solvent such as chloroform. A lipid soluble drug can be co-dissolved at this stage. The mixture can be filtered to remove insoluble matter and the solvent then removed under conditions of temperature and pressure that result in the formation of a dry lipid film. This film is then hydrated using an aqueous medium that can contain hydrophilic compounds to include a drug substance. This hydration process can be controlled so as to control the nature of the resultant liposome formed. When hydration occurs under hand shaking multilamellar liposomes normally result. Smaller liposomes can be produced by the use of sonication and high pressure homogenisation. A French pressure cell can also be used as described by Hamilton and Guo in Liposome Technology, Vol. 1. Gregoriadis, Editor, CRC Press, 1984, p. 37.

Other methods that employ the injection of water immiscible solvents such as ether containing the lipids into an aqueous phase have been described. Other methods include the detergent dialysis method, reverse phase evaporation and extrusion processes. The selection of the method of preparation to provide good drug retention. trapping efficiency has been well described in the prior art (see Betageri et al) thereby allowing the person skilled in the art to prepare a liposome system appropriate to the needs of a particular drug. The present invention is especially useful for drugs that have poor water solubility and would therefore be incorporated into the liquid layer of the liposomal system. The addition of Vitamin E leads to an enhanced level of incorporation.

Preferred features of the invention will now be described in more detail in the following Examples and Figures wherein.

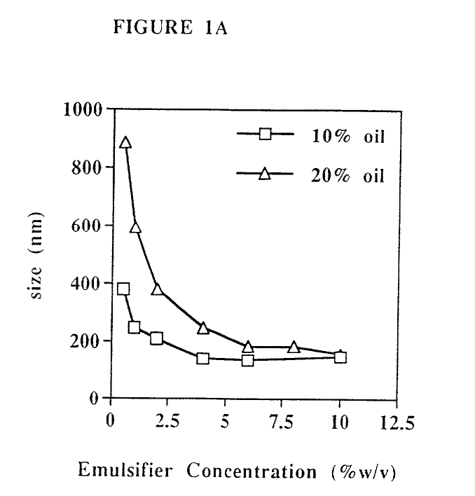

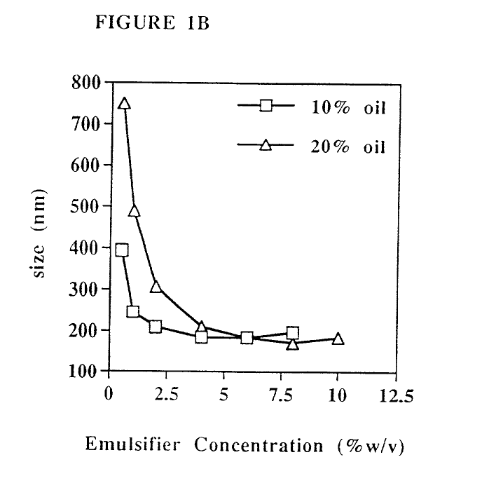

FIG. 1 shows the effect of emulsifier concentration on the emulsion particle size. The oil phase was a mixture of Vitamin E and soybean oil in weight ratio 1:1. A. Pluronic P105 B. Pluronic F127.

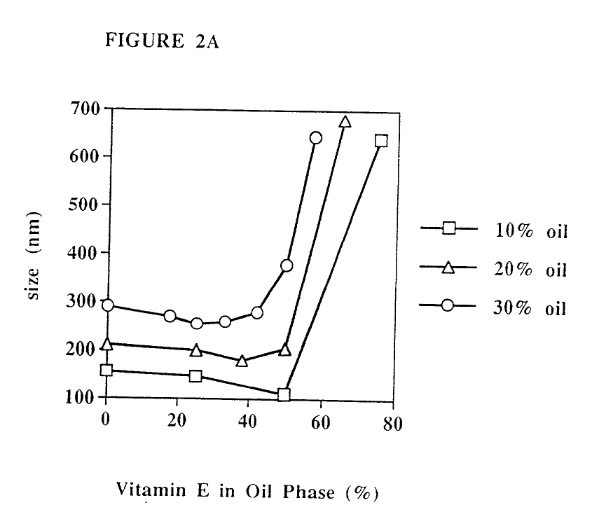

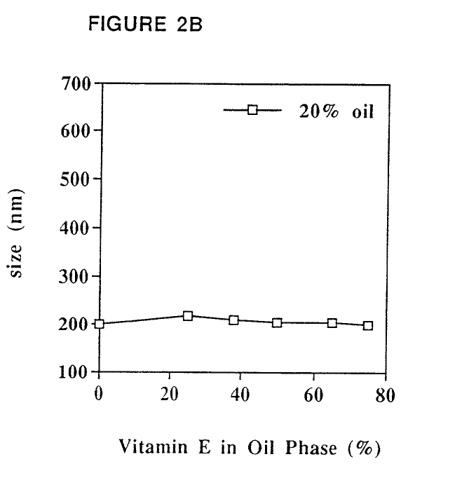

FIG. 2 shows the effect of the amount of Vitamin E in oil phase on the particle size of the emulsion. The other component of the oil phase was soybean oil. The concentration of the emulsifier was 4% (w/v) and it was Pluronic P105 (A) or Pluronic F127 (B).

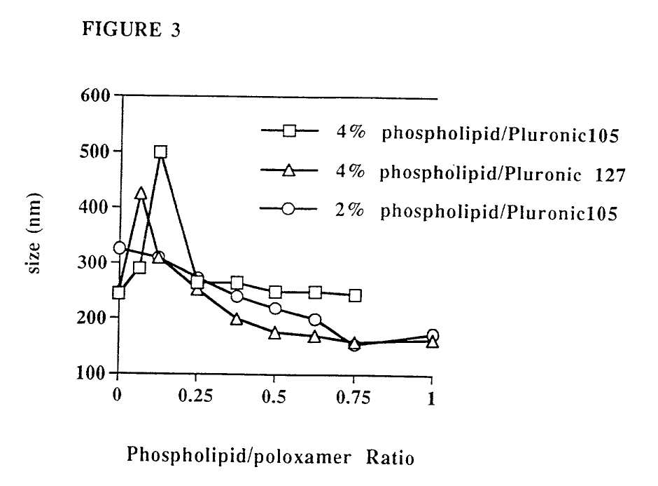

FIG. 3 shows the particle size as a function of the weight ratio of the surfactant (Pluronic P105 or Pluronic F127) and phosphatidylcholine in their mixture used as the emulsifier. The oil phase was 10% v/v and it was composed of Vitamin E and soybean oil in weight ratio 1: 1.

Emulsions of candidate drugs in emulsion formulations were prepared using standard processes such as homogenisation using a microfluidizer (Microfluidic Corporation) or an ultrasonic probe (Dawe, Branson Soniprobe system). The emulsions were prepared by dissolving the drug in the mixture of oil and Vitamin E (or Vitamin E alone), adding the aqueous phase and then preparing a pre-emulsion by use of a high speed stirrer followed by homogenisation using the microfluidizer or ultrasonic method. The aqueous phase of the emulsion contained the dissolved (non-ionic surfactant) or dispersed (phospholipid emulsifier). Emulsions were characterised by means of laser diffractometer (Malvern Mastersizer) and photon correlation spectroscopy (Malvern 4900). Terminal sterilization was achieved using a rotating bench autoclave that could handle 5 ml samples under nitrogen (120° C. 15 psi (103 KPa) for 10 mins).

### EXAMPLE 1

Emulsion 1

| | |
|---|---|
| Soybean oil | 15% |
| Myvacet oil (acetylated monoglycerides) | 15% |
| Phospholipid emulsifier (Epikuron 200 SH) | 4% |
| Vitamin E | 5% |
| Water to | 100% (81 ml) |
| Itraconazole loading | 1.5 mg/ml |

The oils (myvacet and soybean oil) and Vitamin E together with itraconazole were mixed and heated to 60° C. until the solution became clear. Separately the aqueous phase containing the phospholipid emulsifier was heated to 60° C. and blended by stirring at 700 RPM for 2 minutes. The oil phase was then added to the aqueous mix at a rate of 30 ml/min and the resultant mix blended at 13,500 RPM for a further 10 minutes. This formed the pre-emulsion which was then passed through a microfluidizer at 1500 psi for 6 cycles through the machine. The exit temperature was 30-40° C.

### EXAMPLE 2

Emulsion 2

| | |
|---|---|
| Soybean oil | 30% |
| Phospholipid emulsifier (Epikuron 200 SH) | 4% |
| Vitamin E | 5% |
| Water to | 100% (7 ml) |
| Itraconazole loading | 1.4 mg/ml |

The emulsion was prepared as in Example 1 and the mixture sonicated (30 sec on/off cycle) for 5 mins. on a Microson system at a power of 50%.

### EXAMPLE 3

Emulsion 3

| | |
|---|---|
| Soybean oil | 15% |
| Myvacet oil | 15% |
| Epikuron 200 SH | 4% |
| DMPG (dimyristoyl phosphatidyl glycerol) | 0.05% |
| Vitamin B | 5% |
| Water to | 100% (60 ml) |
| Itraconazole loading | 1.5 mg/ml |

The emulsion was prepared as in Emulsion 1. A stable emulsion resulted.

### EXAMPLE 4

Emulsion 4

| | | |
|---|---|---|
| Soybean oil | 1 | ml |
| Vitamin E | 0.25 | ml |
| Egg yolk phospholipid | 200 | mg |
| Water to | 3 | ml |
| Itraconazole loading | 1.7 | mg/ml |

The emulsion was prepared as in Emulsion 1. A stable emulsion resulted.

### EXAMPLE 5

Liposome 1

| | |
|---|---|
| Phospholipid (Epikuron 200 SH) | 740 mg |
| Vitamin E | 198 mg |
| Water | 5 ml |
| Itraconazole loading | 2 mg/ml |

Phospholipid, Epikuron 200 SH, Vitamin E and itraconazole were dissolved in a mixture of chloroform:methanol (2:1). The solvent was removed by thin film evaporation. This was then rotary evaporated until the solvent was removed and the lipid film hydrated with water (5 ml). This solution was then heated to 60° C. and allowed to cool. The itraconazole content was 2.0 mg/ml.

A further series of experiments has been conducted to investigate different emulsion formulations where the amount of Vitamin E, the type of oil and emulsifier (phospholipid or non-ionic surfactant) have been varied. It is clear from these experiments that a person skilled in the art could undertake similar studies in order to select the optimum system for a particular application.

When mixed with soybean oil, (Vitamin E is freely soluble in vegetable oil) emulsions were obtained using egg yolk phospholipid (4%) as emulsifier when the ratio of Vitamin E to vegetable oil was less than 1:2.

Emulsification of undiluted Vitamin E was achieved using a non-ionic surfactant (Poloxamer 188) as emulsifier. Furthermore various mixtures of Vitamin E and vegetable oils were all well emulsified by Poloxamer 118. Some relevant examples are given below by way of illustration.

### EXAMPLE 6

Emulsion 5

To investigate whether a non-ionic emulsifier can be used instead of a phospholipid emulsifier to prepare Vitamin E for use in the invention.

| | |
|---|---|
| Poloxamer 188 | 2% |
| Soybean oil | 20% |
| Vitamin E | 10% |
| Water to | 10 g |

The emulsion was prepared by sonication as for Emulsion 2. A stable emulsion was prepared, mean size 227 nm.

### EXAMPLE 7

Emulsion 6

To investigate whether Vitamin E emulsions could be prepared without the addition of vegetable oil using phospholipid as the emulsifier.

| | |
|---|---|
| Vitamin E | 10% or 50% |
| Egg yolk phospholipid | from 0.4-4% |
| Water | to 10 g |

Stable emulsions were not formed.

### EXAMPLE 8

Emulsion 7

To investigate whether Vitamin E can be emulsified by Poloxamer 188 without the addition of vegetable oil using the sonification procedure as employed for Emulsion 2.

| | |
|---|---|
| Vitamin E | 5% |
| Poloxamer 188 | 0.2% |
| Water | to 10 g |

An emulsion was formed that had a mean size of 630 nm.

### EXAMPLE 9

Self-emulsifying oral formulation 1

500 mg of itraconazole was added to 3600 mg of Vitamin E. The mixture was warmed to 60° C. and stirred until the itraconazole had dissolved. The solution of itraconazole in Vitamin E was cooled to room temperature and 900 mg of polysorbate (Tween) 80 added. Injection moulded starch capsules (Capill, Capsugel) were each filled with 500 mg of the mixture. Thus each capsule contained 50 mg itraconazole, 360 mg of Vitamin E and 90 mg of polysorbate 80.

### EXAMPLE 10

Preparation of emulsions with different oil content

To investigate the effect of total oil content (1:1 ratio of Vitamin E to soybean oil) on emulsion formation and particle size. Emulsions were prepared using the ultrasonic method (output 30%, 5 minutes sonication time). The emulsifier was a poloxamer 407 (Pluronic 127) at 2% w/v concentration. Table 2 shows the effect on particle size and size polydispersity of increasing the oil content in the emulsion when the oil phase is composed of equal weight ratios of soya oil and Vitamin E. Emulsions could be prepared with up to 30% v/v of oil.

TABLE 2
Particle size of emulsions with different oil content. The oil phase was
composed of soya oil and Vitamin E in weight ratio 1:1 and the
emulsifier was Pluronic-127 at 2% (w/v).

| | Size | |
|---|---|---|
| Sample | nm | Polydispersity |
| 5% oil | 168.5 ± 6.6 | 0.299 ± 0.058 |
| 10% oil | 201.5 ± 3.6 | 0.136 ± 0.076 |
| 15% oil | 251.6 ± 2.4 | 0.148 ± 0.040 |
| 20% oil | 306.0 ± 27.6 | 0.348 ± 0.126 |
| 30% oil | 462.2 ± 54.1 | 0.480 ± 0.069 |

### EXAMPLE 11

Preparation of emulsions using Spans and Tweens

To investigate the effect of different emulsifiers. Satisfactory emulsions could be prepared using Polysorbate 20, 40, 60 (Tween 20, Tween 40 and Tween 60) as emulsifiers. Emulsions were prepared by the sonication method as in example 10, at either 10 or 20% v/v oil phase comprising a 1:1 ratio Vitamin E:Soybean oil (w/w). The emulsifier concentration was 4% w/v in all cases. No satisfactory emulsions could be prepared with Polysorbate 65 (Tween 65) and Sorbitan laurate (Span 20). Thus, it may be concluded that the HLB (hydrophilic-lipophilic balance) of the emulsifier should, at least in this system. be greater than 11 (Table 3).

TABLE 3
Particle size of emulsions prepared using different emulsifiers. The oil
phase was soya oil, Vitamin E 1:1 (w/w) and the emulsifier
concentration was 4% (w/v)

| | Size | | |
|---|---|---|---|
| Sample | nm | Polydispersity | HLB |
| Tween 20 10% oil | 151.6 ± 1.4 | 0.243 ± 0.022 | 16.7 |
| Tween 20 10% oil | 166.8 ± 1.8 | 0.218 ± 0.021 | |
| Tween 20 20% oil | 247.4 ± 4.1 | 0.290 ± 0.039 | |
| Tween 40 10% oil | 212.6 ± 4.0 | 0.264 ± 0.034 | 15.6 |
| Tween 40 20% oil | 285.8 ± 4.2 | 0.307 ± 0.041 | |
| Tween 80 10% oil | 194.4 ± 2.9 | 0.360 ± 0.028 | 15 |
| Tween 80 20% oil | 341.0 ± 10.4 | 0.377 ± 0.082 | |
| Tween 65 10% oil | no emuls. | | 10.5 |
| Tween 65 20% oil | > > | | |
| Span 20 10% oil | no emuls. | | 8.6 |
| Span 20 20% oil | > > | | |

### EXAMPLE 12

Preparation of emulsions using poloxamers and poloxamines

To investigate the effect of different block copolymers as emulsifiers. Emulsions were prepared by the sonication method as in example 10, using 10 or 20% v/v oil phase comprising a 1:1 ratio (w/w) of Vitamin E to Soybean oil. The block copolymers were used as emulsifiers at a concentration of 4% v/v. Satisfactory emulsions could be prepared using surfactants of the poloxamer and poloxamine series (block copolymers of polyoxyethylene and polyoxypropylene, commercially available from Wyandotte Chemical as Pluronics and Tetronics). Particle size and polydispersity data are given in Table 4.

TABLE 4
Particle size of emulsions prepared using different poloxamers
(Pluronic) and poloxamines (Tetronic). The oil phase was composed
of soya oil and Vitamin E in weight ratio 1:1 and the concentration
of the various emulsifiers was 4% (w/v).

| | Size | |
|---|---|---|
| Sample | nm | Polydispersity |
| Pluronic L35 10% oil | 147.8 ± 1.8 | 0.118 ± 0.031 |
| Pluronic F127 10% oil | 188.4 ± 2.7 | 0.154 ± 0.041 |
| Pluronic F127 20% oil | 230.8 ± 4.2 | 0.116 ± 0.077 |
| Pluronic L44 10% oil | 154.8 ± 2.6 | 0.146 ± 0.046 |
| Pluronic P105 10% oil | 132.7 ± 1.1 | 0.158 ± 0.036 |
| Pluronic P105 20% oil | 212.0 ± 4.7 | 0.239 ± 0.062 |
| Tetronic 704 10% oil | 155.8 ± 3.8 | 0.252 ± 0.021 |
| Tetronic 904 10% oil | 154.2 ± 5.2 | 0.206 ± 0.041 |
| Tetronic 904 20% oil | 258.7 ± 3.5 | 0.244 ± 0.038 |
| Tetronic 1104 10% oil | 193.2 ± 5.5 | 0.254 ± 0.044 |
| Tetronic 1104 20% oil | 321.6 ± 3.7 | 0.436 ± 0.018 |
| Tetronic 1504 10% oil | 137.6 ± 1.5 | 0.185 ± 0.032 |
| Tetronic 1504 20% oil | 261.4 ± 5.4 | 0.282 ± 0.054 |

### EXAMPLE 13

The effect of surfactant concentration

To investigate the effect of emulsifier concentration. Emulsions were prepared by sonication as in example 10, using a 10 or 20% v/v oil phase content comprising a 1:1 w/w ratio of Vitamin E to Soybean oil. The emulsifiers were Pluronic 105 and Pluronic 127. The emulsions were prepared at increasing concentration of these two emulsifiers from 0 to 10% w/v. It can be seen from FIG. 1 that above 2.5% emulsifier content, no special advantage is gained by the addition of more emulsifier.

### EXAMPLE 14

The effect of Vitamin E in the oil phase

To investigate the effect of combining a block copolymer and phospholipid emulsifier. Vitamin E is not an easy material to emulsify and therefore a preferred embodiment of the invention is to mix the Vitamin E with a vegetable oil such as Soya bean oil, olive oil, sesame oil, caster oil, peanut oil, corn oil. Emulsions were prepared by the ultrasonication method described in Example 10. The Vitamin E content of the oil phase was increased from 0 to 60% w/w. For Pluronic 105, the total oil content of the emulsions was at 10, 20 and 30% v/v. Poloxamines were used as emulsifiers at 4% w/v concentration. For Pluronic 127 the total oil content was 20 % v/v. The properties of the emulsion (particularly particle size) were measured. It can be seen from FIG. 2 that a 50:50 (1:1) mixture of Vitamin E to Soya oil was the limit for a satisfactory emulsion when the emulsifier was Pluronic P105. When using Pluronic F127, suitable emulsions could be at higher contents of Vitamin E in the oil phase.

### EXAMPLE 15

The effect of a combined emulsifier of phospholipid and pluronic block copolymer

To investigate the effect of combining a block copolymer and phospholipid emulsifier. A series of emulsions was prepared wherein the emulsifier was a mixture of hydrogenated egg phospholipid with poloxamer surfactants. The concentration of the phospholipid was held constant at 2 or 4% w/v and Pluronic 105 and F127 added in increasing amounts. The particle size results are given in FIG. 3. The addition of the poloxamers had a beneficial effect on particle size when the weight ratio of poloxamer to phospholipid was greater than 0.3.

### EXAMPLE 16

Emulsions containing Taxol

Taxol can be solubilized into Vitamin E up to a concentration of 40 mg/g dependent on temperature. Taxol was dissolved in Vitamin E in concentrations 10, 20 and 30 mg/g and emulsions with 10% v/v oil phase (Vitamin E, Soybean oil 1:1 w/w ratio) were prepared using different emulsifiers using the sonication method so that the initial concentration of the drug in the emulsion was 0.5, 1 and 1.5 mg/ml, respectively. The measurements of the incorporation efficiency were performed one day after the preparation. The results, which are summarized in Table 5, showed that the incorporation of taxol was lowered when the initial amount of the drug in the oil phase was increased. The determination of the drug concentration in the oil phase showed that with Tetronic 1504, the emulsifier with the highest molecular weight had an advantage over Pluronic P105 since it resulted in a more efficient incorporation of taxol in the emulsion.

TABLE 5
Physical characteristics and taxol incorporation efficiency for
emulsions containing 10% v/v oil phase (Vitamin E, Soybean
oil 1:1 w/.w)

| | Initial taxol | | | % taxol |
|---|---|---|---|---|
| | concentra- | Size | | incorpor- |
| Emulsifier | tion (mg/ml) | (nm) | Polydispersity | ation |
| Pluronic P105 | 0.5 | 122.6 ± 1.2 | 0.204 ± 0.019 | 100 |
| (4% w/v) | 1.0 | 129.4 ± 1.9 | 0.174 ± 0.020 | 41 |
| > > | 1.5 | 126.0 ± 2.6 | 0.187 ± 0.048 | 13 |
| Tetronic 1504 | 0.5 | 130.0 ± 1.3 | 0.134 ± 0.028 | 88 |
| (4% w/v) | 1.0 | 131.3 ± 2.0 | 0.146 ± 0.026 | 61 |
| > > | 1.5 | 134.0 ± 1.1 | 0.142 ± 0.014 | 49 |
| PC Pluronic | 0.5 | 185.3 ± 7.8 | 0.225 ± 0.082 | 46 |
| F127 | | | | |
| (1.25% w/v) | 1.0 | 177.2 ± 4.6 | 0.243 ± 0.083 | 30 |
| > > | 1.5 | 179.4 ± 4.6 | 0.219 ± 0.043 | 37 |
| PC Pluronic | 0.5 | 177.4 ± 4.0 | 0.225 ± 0.044 | 40 |
| F108 | | | | |
| (1.25% w/v) | 1.0 | 172.6 ± 3.9 | 0.210 ± 0.38 | 51 |
| > > | 1.5 | 181.2 ± 3.7 | 0.214 ± 0.025 | 35 |

When the oil content of the emulsion increased to 20% v/v, the incorporation of taxol was improved (Tables 6 and 7). The nature of the material used as emulsifier affected the percentage of the drug that was finally incorporated in the emulsion droplets. More hydrophilic emulsifiers, such as Pluronic F127 and Pluronic F108, gave better results.

TABLE 6
Physical characteristics and taxol incorporation efficiency for
emulsions containing 20% oil phase (vitamin E, soybean oil
0:6:1 w/w). The concentration of the emulsifier was 4% (w/w).

| | Initial taxol | | | % taxol |
|---|---|---|---|---|
| | concentra- | Size | | incorpora- |
| Emulsifier | tion (mg/ml) | (nm) | Polydispersity | tion |
| Pluronic P105 | 2.25 | 190.6 ± 1.8 | 0.097 ± 0.029 | 72 |
| Pluronic F127 | 2.25 | 201.9 ± 2.0 | 0.085 ± 0.037 | 79 |
| Pluronic F108 | 2.25 | 203.0 ± 1.9 | 0.081 ± 0.017 | 81 |
| Tetronic 1504 | 2.25 | 205.0 ± 2.5 | 0.108 ± 0.036 | 62 |

TABLE 7
Physical characteristics and taxol incorporation efficiency for
emulsions containing 20% oil phase (vitamin E, soybean oil 1:1
w/w). The concentration of the emulsifier was 4% (w/v).

| | Initial taxol | | | % taxol |
|---|---|---|---|---|
| | concentra- | Size | | incorpora- |
| Emulsifier | tion (mg/ml) | (nm) | Polydispersity | tion |
| Pluronic P105 | 3.0 | 200.1 ± 2.6 | 0.118 ± 0.026 | 80 |
| Pluronic F127 | 3.0 | 197.6 ± 3.0 | 0.141 ± 0.050 | 82 |
| Tetronic 1307 | 3.0 | 199.0 ± 2.6 | 0.149 ± 0.036 | 56 |

Emulsifiers

Trade and approved names.

Tetronic≡poloxamine (polyethylene oxide, polypropylene oxide block copolymers based on ethylenediamine)

Pluronic≡poloxamer (polyoxyethylene oxide, polypropylene oxide block copolymers)

Pluronic≡Poloxamer

| | |
|---|---|
| P105 | 335 |
| F127 | 407 |
| F108 | 338 |
| L35 | 105 |
| L44 | 124 |

1. A drug delivery composition comprising a lipid vehicle containing drug and Vitamin E to enhance the solubility of the drug in the lipid vehicle, wherein the Vitamin E is present in a concentration of at least 1% in the disperse phase of the lipid vehicle.

2. A drug delivery composition according to claim 1 wherein the lipid vehicle is a liposome or an emulsion system.

3. A drug delivery composition according to claim 1 or 2 wherein the drug has a solubility of at least 1 mg/ml in Vitamin E.

4. A drug delivery composition according to claim 1 or 2 wherein the drug has a solubility in chloroform of 6 mg/ml or more.

5. A drug delivery composition according to claim 4 wherein the ratio of the respective solubility of the drug in chloroform and methanol is greater than 10.

6. A drug delivery composition according to any one of the preceding claims wherein the composition is an oil-in-water emulsion system and the Vitamin E is present in a concentration of at least 1% in the disperse phase of the emulsion.

7. A drug delivery composition according to claim 6 wherein the emulsion additionally comprises a pharmaceutically acceptable oil, and wherein the Vitamin E is provided as a mixture with the pharmaceutically acceptable oil.

8. A drug delivery composition according to claim 6 or 7 wherein the emulsion further comprises an emulsifying agent.

9. A drug delivery composition according to any one of the preceding claims, wherein the drug is an anti-fungal agent, an anti-cancer agent, a retinoid or a steroid.

10. A drug delivery composition according to anyone of claims 1 to 8, wherein the drug is itraconazole, pregnanolone, taxol or a derivative thereof or cyclosporin.

11. A method of administering a drug to a patient the method comprising administering to the patient a drug delivery composition according to any one of the preceding claims.

12. A method according to claim 11 wherein the drug is administered by injection or by administration to any of the eye, the nose, the lung, the gastrointestinal tract, the rectum or the vagina.

13. Use of Vitamin E in the manufacture of a drug delivery composition as defined in any one of claims 1 to 10.

14. A method of making a drug delivery composition according to any one of claims 1 to 12 comprising admixing the lipid vehicle, the active drug and Vitamin E.
